(19) United States
(12) Patent Application Publication
(10) Pub. No.: US 2010/0094100 A1
(43) Pub. Date: Apr. 15, 2010

(54) IMAGE DIAGNOSIS APPARATUS, IMAGE PROCESSING APPARATUS, AND COMPUTER-READABLE RECORDING MEDIUM RECORDING PROGRAM

(76) Inventors: Tomokazu Fujii, Nasushiobara-shi (JP); Kazutoshi Sadamitsu, Otawara-shi (JP); Yoichi Ogasawara, Nasushiobara-shi (JP); Yoshihiro Oomori, Otawara-shi (JP); Satoshi Matsunaga, Nasushiobara-shi (JP)

Correspondence Address: OBLON, SPIVAK, MCCLELLAND MAIER & NEUSTADT, L.L.P., 1940 DUKE STREET, ALEXANDRIA, VA, 22314, US

(21) Appl. No.: 12/574,969

(22) Filed: Oct. 7, 2009

(30) Foreign Application Priority Data
Oct. 14, 2008 (JP) 2008-265383
Oct. 1, 2009 (JP) 2009-229346

(51) Int. Cl. A61B 5/00

(52) U.S. Cl. 600/300

(57) ABSTRACT

An operating image storage unit stores a plurality of codes and a plurality of operation image data sets in association with each other. The plurality of codes is concerned with a plurality of standard user operations. The plurality of operation image data sets is displayed for assisting the plurality of user operations. An operation unit accepts a current user operation by a user. A procedure display engine specifies a specific code concerning a user operation next to the current user operation among the plurality of codes in accordance with an examination procedure for current medical examination. And the procedure display engine specifies a specific operation image data set among the plurality of operation image data sets. The specific operation image data set is concerned with the specific code. A display unit displays the specific operation image and the medical image on the same screen.

### CROSS-REFERENCE TO RELATED APPLICATIONS

This application is based upon and claims the benefit of priority from prior Japanese Patent Applications No. 2008-265383, filed Oct. 14, 2008; and No. 2009-229346, filed Oct. 1, 2009, the entire contents of both of which are incorporated herein by reference.

### BACKGROUND OF THE INVENTION

1. Field of the Invention

The present invention relates to an image diagnosis apparatus, an image processing apparatus, and a computer-readable recording medium recording a program, which are used for medical examination.

2. Description of the Related Art

There are a variety of functions used for medical examination such as ultrasonic examination. In addition, optimal operations differ depending on patients' diseases and their regions. For this reason, many functions which an image diagnosis apparatus has are combined to perform diagnosis in accordance with the condition of the disease of a patient. For example, an ultrasonic diagnosis apparatus has a general B-mode display function for checking a tissue characterization, a color Doppler display function for checking the state of a blood vessel (blood flow), and various measurement functions for checking the diameters and areas of tumors and the like. In ultrasonic examination, the B-mode display function, the color Doppler display function, and the various measurement functions are combined.

An ultrasonic apparatus has the merit of causing no radiation exposure. On the other hand, an ultrasonic probe generates sound waves by using a high voltage. For this reason, the ultrasonic probe generates heat at its portion contacting a living body.

In order to avoid problems at the time of various operations described above, an ultrasonic diagnosis apparatus displays warning messages and messages about operation instructions such as a help instruction. This makes it possible for the ultrasonic diagnosis apparatus to accurately inform an examiner of a situation currently occurring in the apparatus.

Jpn. Pat. Appln. KOKAI Publication No. 11-332865 discloses a technique of detecting the abnormal cardiac rate of a patient at the time of ultrasonic examination and displaying a warning statement or the like on a monitor.

The technique disclosed in Jpn. Pat. Appln. KOKAI Publication No. 11-332865 is to generate a warning statement upon detection of the abnormal cardiac rate of a patient but does not inform a user of many situations occurring in the process of ultrasonic diagnosis.

Ultrasonic diagnosis allows noninvasive real-time observation of the inside of a human body. In addition, ultrasonic diagnosis can diagnose a patient in a short period of time. Owing to these advantages, the number of patients subjected to ultrasonic examination per day has recently being increasing. Furthermore, ultrasonic examination can be applied to many possible examination regions. Examination regions differ depending on the symptoms of patients. Because of these factors, an examiner daily examines various examination regions of many patients.

Under these circumstances, an ultrasonic diagnosis apparatus may encounter unintended problems in various situations. Assume that there is a specific function which cannot be used while a specific operation is performed. An examiner sometimes performs an operation to use this specific function in spite of the situation where he/she is performing this specific operation. In such a case, the ultrasonic diagnosis apparatus displays a warning or a problem. However, the examiner needs to perform a special operation to solve or avoid such a problem. This may stop the process of ultrasonic examination, greatly impairing examination efficiency.

The ultrasonic diagnosis apparatus has many diagnosis tools. The examiner needs to perform many operations or complicated operations for optimal examination. This impairs examination efficiency. When, for example, performing ultrasonic examination using a combination of the B-mode display function, the color Doppler display function, and various measurement functions, the examiner operates many switches on the operation panel. Operation methods and operation procedures for operation panels differ depending on makers and examination contents. For this reason, the examiner must often operate the operation panel while checking it and looking aside from an examination region of a patient. Such complexity of operation impairs examination efficiency.

### BRIEF SUMMARY OF THE INVENTION

It is an object of the present invention to provide an image diagnosis apparatus, an image processing apparatus, and a computer-readable recording medium recording a program, which can improve examination efficiency.

According to a first aspect of the present invention, an image diagnosis apparatus includes: a generating unit configured to generate a medical image data set for medical examination; a first storage unit configured to store a plurality of codes and a plurality of operation image data sets in association with each other, the plurality of codes being concerned with a plurality of standard user operations in the medical examination, the plurality of operation image data sets being displayed for assisting the plurality of user operations; an operation unit configured to accept a current user operation by a user; a first specifying unit configured to specify a specific code concerning a user operation next to the current user operation among the plurality of codes in accordance with an examination procedure for current medical examination corresponding to the current user operation; a second specifying unit configured to specify a specific operation image data set among the plurality of operation image data sets, the specific operation image data set being concerned with the specific code; and a display unit configured to display the specific operation image and the medical image on the same screen.

According to a second aspect of the present invention, an image processing apparatus includes: a storage unit configured to store a plurality of codes and a plurality of operation image data sets in association with each other, the plurality of codes being concerned with a plurality of standard user operations in medical examination, the plurality of operation image data sets being displayed for assisting the plurality of user operations; an operation unit configured to accept a current user operation by a user; a first specifying unit configured to specify a specific code concerning a user operation next to the current user operation among the plurality of codes in accordance with an examination procedure for current medical examination corresponding to the current user operation; a second specifying unit configured to specify a specific operation image data set among the plurality of operation image data sets, the specific operation image data set being concerned with the specific code; and a display unit configured to display the specific operation image.

According to a third aspect of the present invention, a computer-readable recording medium recording a program for causing a computer to: accept a current user operation, specify a code concerning a user operation next to the current user operation among a plurality of codes concerning a plurality of user operations in accordance with an examination procedure for medical examination corresponding to the current user operation, specify a operation image data set concerning the specified code among a plurality of operation information files representing the plurality of user operations, and display the specified operation image.

Additional objects and advantages of the invention will be set forth in the description which follows, and in part will be obvious from the description, or may be learned by practice of the invention. The objects and advantages of the invention may be realized and obtained by means of the instrumentalities and combinations particularly pointed out hereinafter.

### BRIEF DESCRIPTION OF THE SEVERAL VIEWS OF THE DRAWING

The accompanying drawings, which are incorporated in and constitute a part of the specification, illustrate embodiments of the invention, and together with the general description given above and the detailed description of the embodiments given below, serve to explain the principles of the invention.

FIG. 1 is a block diagram showing the schematic arrangement of an image diagnosis apparatus (ultrasonic diagnosis apparatus) according to the embodiment of the present invention;

FIG. 2 is a flowchart for explaining the operation of the embodiment;

FIG. 3 is a view showing a display example on a display unit in FIG. 1;

FIG. 4 is a view showing another display example on the display unit in FIG. 1;

FIG. 5 is a view showing still another display example on the display unit in FIG. 1;

FIG. 6 is a view showing still another display example on the display unit in FIG. 1;

FIG. 7 is a flowchart for explaining the operation of a modification of the first embodiment of the present invention; and

FIG. 8 is a view showing a display example on the display unit in the modification of the first embodiment.

### DETAILED DESCRIPTION OF THE INVENTION

An embodiment of the present invention will be described below with reference to the views of the accompanying drawing.

This embodiment supports medical examination performed by an examiner. Medical examination means every operation associated with medical services such as the generation of medical image data set, the interpretation of medical images, the measurement of various measurement parameters on medical images, and the generation of clinical charts. Medical examination is performed in accordance with an examination procedure corresponding to the type of examination. An examination procedure is also called a work flow or protocol. An examination procedure is a sequence of a plurality of user operations for executing the medical examination. User operations are typically operations by an examiner or the like with respect to various electric components mounted on an operation panel.

This embodiment is applied to an image diagnosis apparatus, an image processing apparatus (computer), and a computer-readable recording medium recording a program. The image diagnosis apparatus according to this embodiment includes medical modalities such as an ultrasonic diagnosis apparatus, X-ray diagnosis apparatus, X-ray computer tomography apparatus, magnetic resonance imaging apparatus, and nuclear medicine diagnosis apparatus. For a specific description below, assume that the image diagnosis apparatus is an ultrasonic diagnosis apparatus. Assume also that the image processing apparatus according to this embodiment is a computer incorporated in the ultrasonic diagnosis apparatus.

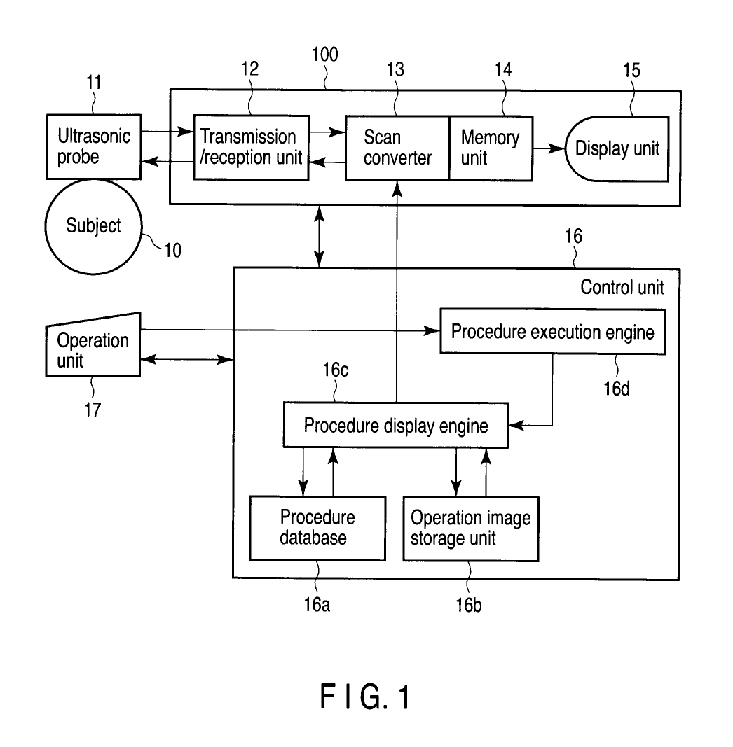

FIG. 1 shows the schematic arrangement of the ultrasonic diagnosis apparatus according to the embodiment of the present invention.

As shown in FIG. 1, the ultrasonic diagnosis apparatus includes an ultrasonic probe 11. The ultrasonic probe 11 transmits/receives ultrasonic waves. An examiner brings the ultrasonic probe 11 into contact with a subject 10 during examination. The ultrasonic probe 11 is connected to an apparatus body 100. The apparatus body 100 includes a transmission/reception unit 12, a digital scan converter 13, a memory unit 14, a display unit 15, and a control unit 16. The transmission/reception unit 12 is connected to the ultrasonic probe 11. The transmission/reception unit 12 functions as a unit to generate an ultrasonic image data set for medical examination. The transmission/reception unit 12 drives the ultrasonic probe 11 to transmit ultrasonic waves, and generates ultrasonic image data set representing the inside of the subject 10 by processing received reflected signals.

The digital scan converter 13 is connected to the transmission/reception unit 12. The digital scan converter 13 includes the memory unit 14. The digital scan converter 13 converts the ultrasonic image data set generated by the transmission/reception unit 12 into a display data set. The memory unit 14 then stores the display data set. The display unit 15 is connected to the digital scan converter 13. The display unit 15 displays the ultrasonic image data set stored in the memory unit 14 as an ultrasonic image.

The display unit 15 also displays various operation images (to be described later). The display unit 15 displays an operation image and an ultrasonic image on the same screen.

A control unit 16 controls the overall apparatus body 100. An operation unit 17 for operating the ultrasonic diagnosis apparatus is connected to the control unit 16. The operation unit 17 has an operation panel. The operation unit 17 accepts a user operation via the operation panel which is performed by the examiner. The control unit 16 instructs the execution of an examination procedure corresponding to an examination region and the like. The control unit 16 determines the current situation of the apparatus (the current situation or problem of the apparatus), when, for example, the examiner cannot perform an intended operation in accordance with the examination procedure. The control unit 16 includes a procedure database 16a, an operation image storage unit 16b, a procedure display engine 16c, and a procedure execution engine 16d. The procedure database 16a stores, for example, a plurality of examination procedures for a plurality of medical examinations. The plurality of examinations differs depending on examination regions. The operation image storage unit 16b stores an operation image data set for assisting a user operation. An operation image data set includes various message image data sets (warnings and instruction messages), a reduced image data set, an illustration image data set, and a component image data set. An operation image data set is one of pieces of operation information. A reduced image data set is an image data set representing a copy of a reduced operation panel. An illustration image data set is an image data set representing an illustration of the operation panel. A component image data set is an image data set concerning electric components mounted on the operation panel. The electric components are required for an operation. A component image data set is an image data set obtained by, for example, extracting some switches on the operation panel. The procedure display engine 16c generates an operation image data set visually expressing an examination procedure stored in the procedure database 16a. The operation image data set generated by the procedure display engine 16c is an image data set concerning, for example, a flowchart. The procedure display engine 16c outputs the generated flowchart image data set to the digital scan converter 13. The procedure display engine 16c stores the generated flowchart image data set in the operation image storage unit 16b. The procedure display engine 16c also outputs various operation image data sets stored in the operation image storage unit 16b to the digital scan converter 13. The digital scan converter 13 converts these outputs from the procedure display engine 16c into display data. The display unit 15 then displays the display data. The procedure execution engine 16d executes processing corresponding to the operation image data set selected by the examiner via the operation panel.

More specifically, the operation image storage unit 16b stores a plurality of codes concerning a plurality of user operations. User operations are standard operations performed in medical examination. For example, user operations include pressing various buttons, switching switches, rotating a dial, operating a mouse, and operating electric components mounted on the operation panel, e.g., operating a probe. Buttons include, for example, a SET key, Freeze button, an image save button, and a print button. Switches include, for example, a Cine_Store switch, a Still_Store switch, and Meas.Edit switch. The operation image storage unit 16b further stores a plurality of user operations and a plurality of operation image data sets in association with each other. For example, the operation image storage unit 16b stores a code (a code concerning a user operation) concerning the press of the image save button and a reduced image data set (operation image data set) of the image save button in association with each other.

In addition, the procedure display engine 16c specifies a code, of the plurality of codes stored in the operation image storage unit 16b, which concerns a user operation next to the current user operation accepted by the operation unit 17 in accordance with an operation procedure for medical examination corresponding to the current user operation. As described above, the procedure display engine 16c has a function of specifying the next user operation. The procedure display engine 16c specifies an operation image data set associated with the code concerning the next user operation among the plurality of operation image data sets stored in the operation image storage unit 16b. As described above, the procedure display engine 16c has a function of specifying an operation image data set.

The operation of the embodiment having such an arrangement will be described next. An example of operation to be performed when an operation image data set is a message image data set will be described first. Assume that medical examination in the following operation example is ultrasonic examination which the examiner performs while operating the ultrasonic probe.

When the transmission/reception unit 12 drives the ultrasonic probe 11, ultrasonic waves are transmitted to the Subject 10. An ultrasonic image data set representing the inside of the Subject 10 is generated based on the reflected signals received by the transmission/reception unit 12. The digital scan converter 13 converts the generated ultrasonic image data set into a display data set. The memory unit 14 stores the display data set. The display unit 15 displays the display data set as an ultrasonic image.

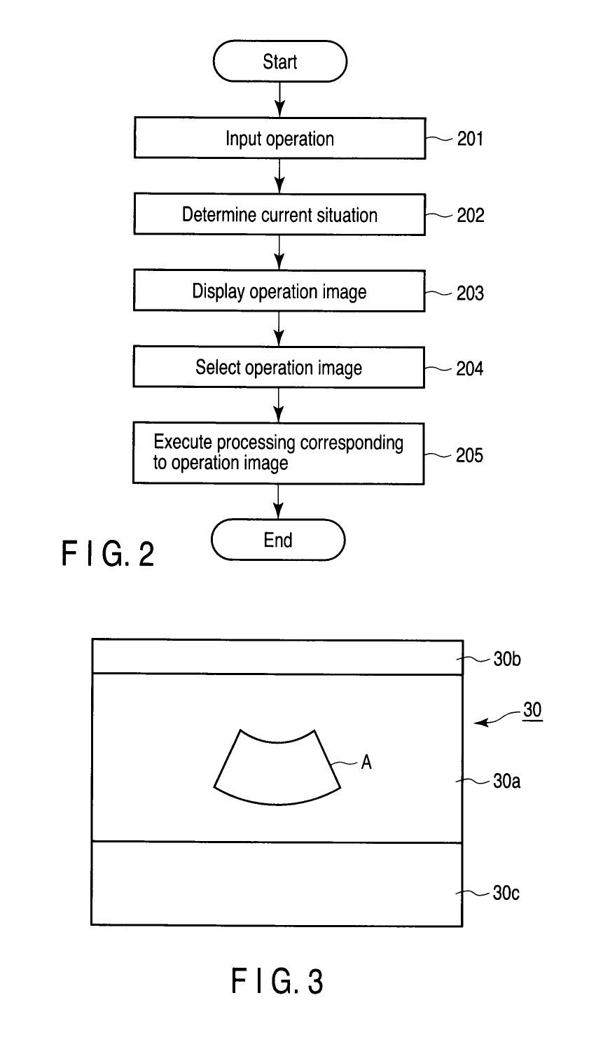

As the examiner performs various user operations on the operation panel, the settings in the ultrasonic diagnosis apparatus change every moment. For example, the examiner switches the B-mode display mode to the Doppler display mode. If the examiner cannot perform a user operation as intended, the control unit 16 executes processing in accordance with the procedure shown in the flowchart of FIG. 2. First of all, in step 201, when the control unit 16 receives, from the examiner via the operation unit 17, information indicating that he/she cannot perform an intended operation, the process advances to step 202. In step 202, the control unit 16 determines the current situation or problem of the apparatus. In step 203, the procedure display engine 16c reads out a message image data set corresponding to the determination result obtained by the control unit 16 from the operation image storage unit 16b. The control unit 16 then causes the display unit 15 to display the readout message image data set via the digital scan converter 13.

FIG. 3 is a view showing an example of a display image 30 displayed on a screen on the display unit 15. As shown in FIG. 3, the display image 30 includes an image display area 30a, a patient information display area 30b, and a message display area 30c. In the image display area 30a, an ultrasonic image A is displayed on the central portion of the display image 30. The patient information display area 30b is displayed above the image display area 30a. Patient information such as a patient ID for specifying the subject 10 is displayed in the patient information display area 30b. The message display area 30c is displayed below the image display area 30a. The above message image is displayed in the message display area 30c.

For example, a message describing a current problem is depicted on a message image. If, for example, the examiner presses the save button in spite of the fact that no patient ID has been input, “image cannot be saved because of no patient ID” is displayed as a message representing the current problem.

When the display unit 15 displays the message image, the examiner selects the message image on the message display area 30c via the operation unit 17. More specifically, the examiner operates the mouse on the operation panel to move the cursor onto the message display area 30c, and inputs the intention to “decide” by operating the SET key or the left switch of the mouse. Inputting this intention to “decide” will select a desired message image via the operation unit 17 (step 204). In step 205, the procedure execution engine 16d specifies a user operation to be executed next in accordance with the contents of the message image displayed in the message display area 30c. The procedure execution engine 16d executes processing corresponding to the specified next user operation. Therefore, the processing is executed by the same operation procedure as when the operation panel is used.

Note that the operation image displayed on the display unit 15 in step 203 is not limited to the message image shown in FIG. 2. For example, the operation image to be displayed includes a reduced image, illustration image or a component image.

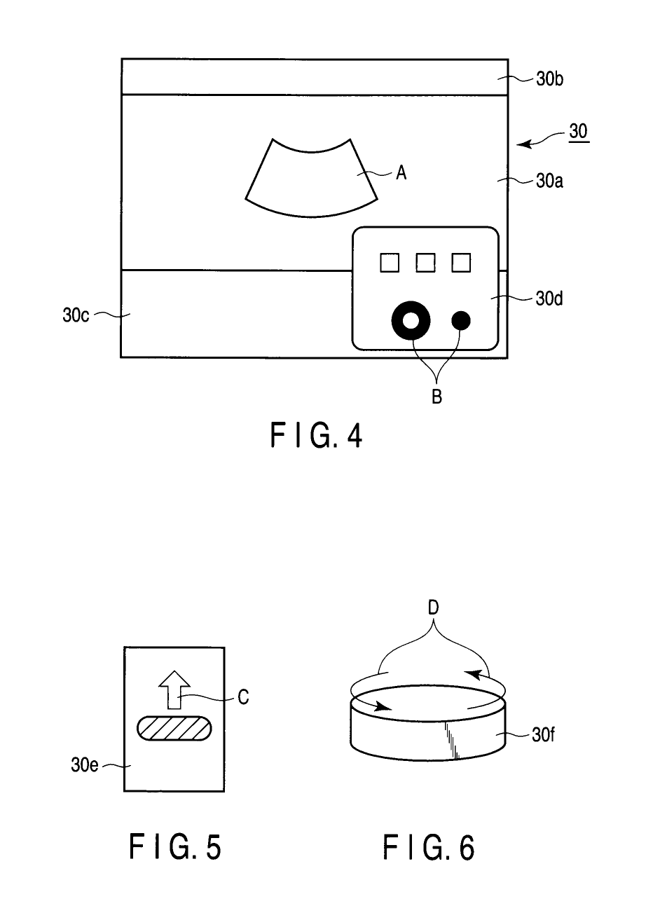

FIG. 4 is a view showing another display example on the display unit 15. As shown in FIG. 4, on the display image 30, an operation panel display area 30d is displayed, in addition to the image display area 30a, the patient information display area 30b, and the message display area 30c described above. A reduced image or illustration image of the operation panel is displayed in the operation panel display area 30d. The procedure display engine 16c reads out a reduced image data set or illustration image data set from the operation image storage unit 16b. The display unit 15 displays, via the digital scan converter 13, the readout reduced image data set or illustration image data set. In this case, the display unit 15 highlights a specific electric component portion on the reduced image or illustration image. For example, the display unit 15 highlights a switch portion which is to attract attention from the examiner, as indicated by “B” in the operation panel display area 30d in FIG. 4. This highlighting allows the examiner to perform an intuitive operation.

In this state, for example, the examiner operates the mouse on the operation panel to select a desired switch portion. More specifically, the examiner moves the cursor on the screen onto the desired switch portion on the operation panel display area 30d, and inputs the intention to “decide” by operating the SET key or the left switch of the mouse. When the desired switch portion is selected via the mouse, the operation panel display area 30d specifies processing corresponding to the selected switch portion and executes the specified processing.

More specifically, the operation image storage unit 16b stores codes concerning a plurality of electric component portions and a plurality of processing contents in association with each other. A processing content is processing assigned to an actual electric component on the operation panel which corresponds to an electric component portion. When an electric component portion is selected via the operation unit 17, the procedure execution engine 16d searches the operation image storage unit 16b by using a code concerning the selected electric component portion as a search keyword. The procedure execution engine 16d then specifies a processing content associated with the selected electric component portion. The procedure execution engine 16d executes processing in accordance with the specified processing content. That is, the procedure execution engine 16d executes the same processing as that assigned to the actual electric component corresponding to the selected electric component portion. Therefore, the processing is executed by the same operation procedure as when the operation panel is used. Note that processing contents are associated with codes concerning electric component portions. However, this embodiment need not be limited to this. For example, the operation image storage unit 16b may store processing contents and the coordinates of electric component portions on a reduced image or illustration image in association with each other.

An example of operation in a case in which an operation image data set is a component image data set will be described next. When, for example, a message image concerning a current problem is displayed, the display unit 15 displays a component image representing a method of solving the current problem abreast of the message image. If, for example, the examiner presses the save button in spite of the fact that no patient ID has been input, the display unit 15 displays a component image concerning a button for displaying a patient ID input window. When the examiner selects a component image via the operation unit 17, the procedure execution engine 16d executes processing corresponding to the selected component image. More specifically, the procedure execution engine 16d executes the same processing as that assigned to the button depicted on the component image.

An example of operation in a case in which an operation image data set is a component image data set will be described next with reference to the flowchart of FIG. 2 described above. As described above, the procedure database 16a stores a plurality of codes concerning a plurality of user operations in medical examination. The procedure database 16a also stores a plurality of standard examination procedures for a plurality of medical examinations. In step 201, the operation unit 17 accepts a current user operation. When the current user operation is input, the procedure display engine 16c analyzes a plurality of examination procedures stored in the procedure database 16a in accordance with the type of current user operation and the order in which the current user operation has been performed. The procedure display engine 16c then specifies a current examination procedure for current medical examination among the plurality of examination procedures.

Every time the examiner performs an operation via the operation unit 17, the current user operation is input (step 201). The procedure display engine 16c determines the current situation or problem of the apparatus in accordance with the input current user operation (step 202). The control unit 16 reads out a component image data set corresponding to the determination result in step 202 from the operation image storage unit 16b (step 203). An example of reading out a component image data set in step 203 will be described below.

In step 203, the procedure display engine 16c specifies a user operation (next user operation) to be performed next to the current user operation in accordance with the specified current examination procedure. The procedure display engine 16c then searches the operation image storage unit 16b by using a code representing the specified next user operation as a search keyword. The procedure display engine 16c specifies a component image data set associated with the next user operation among a plurality of component image data sets. A component image data set is associated with an electric component such as a button, switch, or dial for the next user operation. The display unit 15 displays the specified component image. This component image is displayed to be selectable with the operation unit 17.

When a component image is displayed in step S203, the control unit 16 waits for the selection of a component image via the operation unit 17. When the examiner selects a component image via the operation unit 17 (step 204), the procedure execution engine 16d executes the processing assigned to the actual electric component corresponding to the selected component image (step 205). If, for example, a component image concerning the STORE_STILL button is selected, the procedure execution engine 16d executes the processing assigned to the STORE_STILL button. More specifically, the procedure execution engine 16d saves the ultrasonic image displayed on the display unit 15.

The display unit 15 can present the examiner with a user operation to be performed next to the current user operation by displaying a component image in this manner. As described above, the component image is displayed on the same screen as that of an ultrasonic image. The examiner can therefore check the next user operation without almost averting his/her eyes from the ultrasonic image. When the examiner selects a component image via the operation unit 17, the procedure execution engine 16d executes processing corresponding to the selected component image. The procedure execution engine 16d can therefore execute the next user operation suitable for the current user operation while minimizing the movement of the line of sight of the examiner. In addition, since the processing corresponding to the next user operation is executed, the examiner need not actually operate an electric component on the operation panel. That is, the examiner can execute an optimal user operation while paying attention to the ultrasonic image as much as possible.

Note that the operation image data set in the above operation example is a component image data set. However, the above operation example is not limited to this. That is, the operation image data set according to the above operation example can be any kind of operation image data set such as a message image data set, reduced image data set, or illustration image data set.

FIGS. 5 and 6 are views each showing still another display example on the display unit 15. FIG. 5 shows a component image 30e concerning some switch necessary for an operation on the operation panel. FIG. 6 shows a component image 30f concerning some dial necessary for an operation on the operation panel. Both the component image 30e and the component image 30f are displayed as still images. The procedure display engine 16c reads out the component images 30e and 30f from the operation image storage unit 16b, and displays them on the display unit 15 via the digital scan converter 13. If the examiner wants to emphasize the component images 30e and 30f, the display unit 15 highlights the component images 30e and 30f by the same method as described above. The display unit 15 displays the moving direction of an electric component near the component image. For example, as shown in FIG. 5, if the component image 30e is associated with a switch for an up/down operation, the display unit 15 may display an arrow C indicating an up/down direction together with the component image 30e. In addition, if the component image 30f is associated with a dial for a rotating operation, the display unit 15 may display an arrow D indicating the rotating direction together with the component image 30f.

In this case as well, the examiner operates, for example, the mouse on the operation panel to move the cursor on the display image 30 onto the component image 30e (30f), and inputs the intention to “decide” with the SET key or the left switch of the mouse. When the examiner inputs the intention to “decide” via the mouse, the procedure execution engine 16d specifies a user operation corresponding to the switch or dial operation displayed on the component image 30e (30f), and executes processing corresponding to the specified user operation. Therefore, the procedure execution engine 16d executes the processing by the same operation procedure as when the operation panel is used.

With the above arrangement, if the examiner cannot perform a user operation as intended, the control unit 16 determines the current situation or problem of the apparatus. The control unit 16 then reads out, from the operation image storage unit 16b, an operation image data set such as a message image data set corresponding to the determination result, a reduced image data set of the operation panel, an illustration image data set of the operation panel, or a component image data set. The control unit 16 then displays the readout operation image on the screen, together with the ultrasonic image. In other words, the control unit 16 can display an operation image concerning the next user operation in accordance with the current user operation. When the displayed operation image is selected, the control unit 16 further executes processing corresponding to the selected operation image. Therefore, the control unit 16 can perform processing by the same operation procedure as when the operation panel is used.

With this operation, for example, the examiner can check the message image displayed on the display unit 15 as in a conventional manner, and it is possible to execute an optimal user operation while the examiner keeps paying attention to the examination screen as much as possible. That is, this embodiment can eliminate factors that degrade examination efficiency, e.g., stoppage of the process of examination, thereby dramatically improving the examination efficiency.

In addition, the display unit 15 displays an operation image visually expressing the next user operation (for example, a component image concerning an electric component such as a switch or a button on the operation panel which is necessary for the next user operation, i.e., an image obtained by extracting only an electric component, of the plurality of electric components on the operation panel, which is necessary for the next user operation) to explicitly indicate the user operation to be performed next by the examiner. Displaying the next user operation in this manner allows the examiner to intuitively and easily check the user operation to be performed next. With this function, this embodiment can be expected to smooth the process of examination and improve examination efficiency.

In addition, when an operation image is selected on the display unit 15, the image diagnosis apparatus according to this embodiment can execute processing most suitable for the selected operation image. With this function, the image diagnosis apparatus according to this embodiment can be expected to smooth the process of examination and improve examination efficiency.

In addition, an operation image concerning the next user operation is displayed on the same screen as that of an ultrasonic image. This makes it possible for the examiner to check the next user operation while observing an ultrasonic image. By selecting the displayed operation image, the examiner can execute the next user operation. That is, the examiner need not move his/her line of sight to the operation panel, and is allowed to keep watching the screen. This makes it possible for the image diagnosis apparatus according to this embodiment to smooth the process of examination concerning the generation of image data and improve examination efficiency.

Ultrasonic examination, in particular, requires a complicated operation of an ultrasonic probe. In addition, during ultrasonic examination, the examiner operates the ultrasonic probe while observing an ultrasonic image displayed in real time. Therefore, applying the technique according to this embodiment to ultrasonic examination allows to expect to minimize the movement of the line of sight of the examiner and greatly improve examination efficiency.

In addition, ultrasonic examination has various applications. Processing assigned to each electric component on the operation panel may differ depending on applications. That is, when performing a user operation on the operation panel, the user needs to memorize processing assigned to each electric component for each application. In contrast, this embodiment displays only an electric component necessary for the next user operation as a GUI on the screen. Therefore, the user need not memorize processing assigned to each electric component on the operation panel. These can improve examination efficiency and facilitate examination.

### (Modification)

In general, ultrasonic diagnosis includes different examination procedures in accordance with examination regions. Examination procedures greatly vary depending on examination regions and degrees of diseases. Disease region measurement and fetometry, in particular, include several types of examination procedures even for examination on the same region.

An operation image data set according to the modification is a flowchart image data set visually expressing an examination procedure. As described above, the procedure display engine 16c generates this flowchart image data set.

The procedure database 16a according to the modification stores a plurality of codes concerning a plurality of user operations and a plurality of block image data sets concerning a plurality of user operations in association with each other. A block image data set is an image data set visually expressing each block on a flowchart. A block image data set is one of operation image data sets.

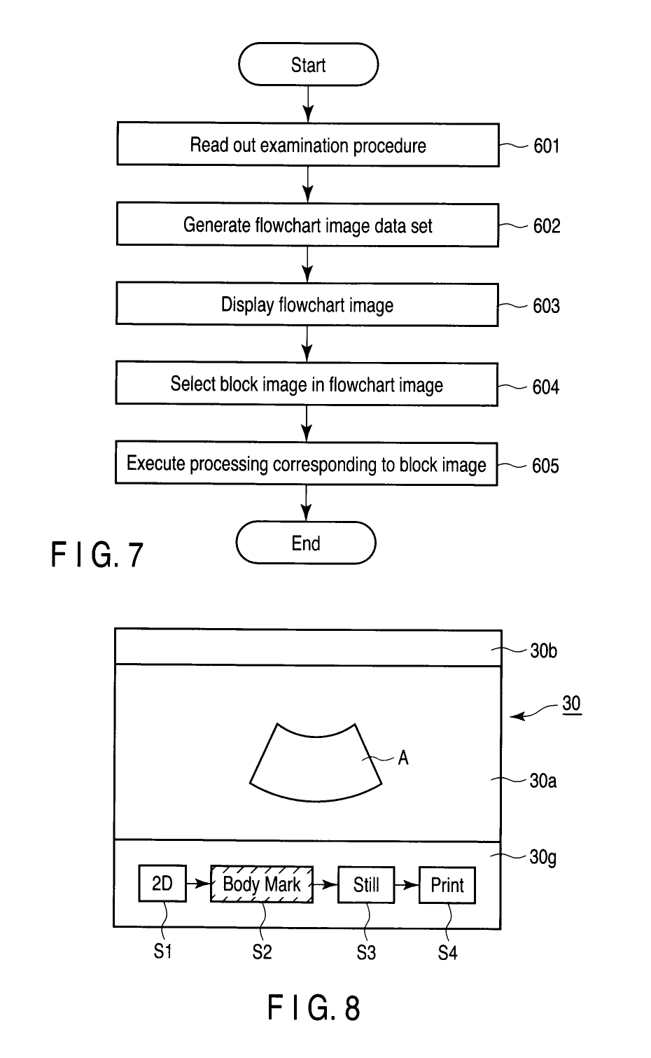

The control unit 16 executes processing in accordance with the procedure shown in the flowchart of FIG. 7. In step 601, the control unit 16 reads out an examination procedure corresponding to examination contents from the procedure database 16a, and inputs the procedure to the procedure display engine 16c. In step 602, the procedure display engine 16c then generates a flowchart image data set in accordance with the examination procedure read out from the procedure database 16a. In step 603, the procedure display engine 16c displays the flowchart image on the display unit 15 via the digital scan converter 13.

The processing from step 601 to step 603 described above will be described in more detail. Every time the examiner performs an operation via the operation unit 17, the current user operation is input. The control unit 16 then analyzes a plurality of examination procedures stored in the procedure database 16a in accordance with the type of an input current user operation and the order in which the current user operation has been performed. The procedure display engine 16c then specifies a current examination procedure for the current medical examination among the plurality of examination procedures. The procedure display engine 16c then reads out the specified current examination procedure from the procedure database 16a (step 601).

Upon reading out the current examination procedure, the procedure display engine 16c reads out a plurality of block image data sets concerning a plurality of user operations constituting the current examination procedure from the operation image storage unit 16b. The procedure display engine 16c then generates a flowchart image data set by arraying the plurality of readout block image data sets in accordance with the examination procedure (step 602). When the flowchart image data set is generated, the display unit 15 displays the generated flowchart image (step 603).

FIG. 8 is a view showing an example of the display image 30 displayed on the screen on the display unit 15. As shown in FIG. 8, the display image 30 includes a flowchart image display area 30g, in which the above flowchart image is displayed, in addition to the image display area 30a and the patient information display area 30b. In the flowchart image display area 30g, a flowchart image including a block image representing step S1, a block image representing step S2, a block image representing step S3, and a block image representing step S4 is displayed. The block image representing step S1 depicts “display one ultrasonic image (2D)”. The block image representing step S2 depicts “display examination region (Body Mark)”. The block image representing step S3 depicts “save still image (Still)”. The block image representing step S4 depicts “print (Print)”.

When the flowchart image is displayed, the examiner selects a desired block image from the flowchart image. For example, the examiner operates the mouse on the operation panel to move the cursor on the screen on the display unit 15 to a desired block image, and confirms to “decide” the block image by operating the SET key or the left switch of the mouse. When the examiner confirms to “decide” the block image, a desired message image is selected via the operation unit 17 (step 604). When the examiner confirms to “decide” the block image via the operation unit 17, the procedure execution engine 16d specifies a user operation to be executed from the contents of the selected block image, and executes processing corresponding to the specified user operation in step 605. As a consequence, the procedure execution engine 16d executes the processing by the same procedure as that of a user operation on the operation unit 17. In this case, the display method for a block image corresponding to executed processing is changed by, for example, highlighting (or color change). In the case shown in FIG. 8, the display method for the block image representing step S2 is changed (the hatched portion in FIG. 8).

Assume that abdominal screening has been executed. In a typical clinical case, the abdominal screening shifts to contrast medium examination or is continued. In this case, the procedure display engine 16c generates a flowchart image data set having a branch point. At the branch point, the flowchart branches into a procedure in which abdominal screening shifts to contrast medium examination and a procedure in which abdominal screen is continued. The display unit 15 displays the generated flowchart image. This makes it possible for the examiner to check the flowchart with reference to an examination order and the procedure of actually performed examination. In addition, selecting a block image in the flowchart image can easily execute the next user operation.

Note that when many block images overflowing the flowchart image display area 30g are displayed, the display unit 15 automatically scrolls a message group as processing corresponding to the block images is executed.

As described above, the image diagnosis apparatus according to the modification can present optimal examination to the examiner by displaying a message group on a screen in accordance with an examination procedure. The examiner then selects a message image of these message groups, and presses a switch to confirm to “decide” the block image with the mouse. As a consequence, the image diagnosis apparatus according to the modification executes processing most suitable for the selected message group. With this operation, the processing is executed by the same procedure as that of a user operation on the operation unit 17. This improves the examination efficiency and quality. In addition, it can be expected to execute examinations with uniform contents regardless of the years of experience or knowledge of the examiner himself/herself.

Note that in the above modification, the operation image data set concerning the examination procedure is a flowchart image data set. However, this embodiment need not be limited to this. For example, the operation image data set concerning the examination procedure can be a set of reduced images of the operation panel stored in the operation image storage unit 16b. In this case, the display unit 15 may display switch portions highlighted on reduced images while arranging them in accordance with an operation procedure. In this case as well, the examiner selects a desired switch portion among the displayed switch portions via the operation unit 17. The procedure execution engine 16d then executes processing most suitable for the selected switch portion. With this operation, the processing is executed by the same procedure as that of a user operation on the operation unit 17. In this case, it is possible to display block images describing operation contents side by side without displaying any reduced image of the operation panel.

The above embodiment has exemplified the image diagnosis apparatus. However, the embodiment can be applied to an image processing apparatus such as a computer or workstation, as described above. The image processing apparatus includes at least the display unit 15, the control unit 16, and the operation unit 17. Note that the image processing apparatus does not have any mechanism for scanning an object. In the case of the image processing apparatus, therefore, “generation of medical image data” is not included in the category of medical examination according to this embodiment.

Each function associated with this embodiment can also be implemented by installing programs for executing the corresponding processing in a computer such as a workstation and mapping them in a memory. In this case, the programs which can cause the computer to execute the corresponding techniques can be distributed by being stored in recording media such as magnetic disks (Floppy® disks, hard disks, and the like), optical disks (CD-ROMs, DVDs, and the like), and semiconductor memories.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An image diagnosis apparatus comprising:
a generating unit configured to generate a medical image data set for medical examination;
a first storage unit configured to store a plurality of codes and a plurality of operation image data sets in association with each other, the plurality of codes being concerned with a plurality of standard user operations in the medical examination, the plurality of operation image data sets being displayed for assisting the plurality of user operations;
an operation unit configured to accept a current user operation by a user;
a first specifying unit configured to specify a specific code concerning a user operation next to the current user operation among the plurality of codes in accordance with an examination procedure for current medical examination corresponding to the current user operation;
a second specifying unit configured to specify a specific operation image data set among the plurality of operation image data sets, the specific operation image data set being concerned with the specific code; and
a display unit configured to display the specific operation image and the medical image on the same screen.

2. The apparatus according to claim 1, wherein the operation image data set includes at least one of a message image data set, a reduced image data set of an operation panel, an illustration image data set of the operation panel, and a component image data set, the message image data set being concerned with at least one of a problem corresponding to the current user operation and a solution for the problem, the component image data set being concerned with an electric component on the operation panel, the electric component being associated with the current user operation.

3. The apparatus according to claim 2, wherein if the specific operation image is a component image, the display unit configured to display a mark indicating a movable range of an electric component corresponding to the component image.

4. The apparatus according to claim 1, wherein the operation image data set is a flowchart image data set schematically representing an examination procedure for medical examination.

5. The apparatus according to claim 4, wherein the flowchart image data set includes a plurality of block image data sets concerning a plurality of blocks on the examination procedure.

6. The apparatus according to claim 1, further comprising an execution unit configured to execute specific processing corresponding to the specific operation image data set, when the specific operation image is selected via the operation unit.

7. The apparatus according to claim 6, which further comprises a second storage unit configured to store the plurality of operation image data sets and the plurality of processing contents in association with each other, and in which
the execution unit configured to search the second storage unit by using the specific operation image data set as a search keyword, specifies a specific processing content associated with the specific operation image data set, and executes the specific processing in accordance with the specific processing content.

8. The apparatus according to claim 1, further comprising
a third storage unit configured to store a plurality of standard examination procedures for a plurality of medical examinations, each of the plurality of examination procedures being constituted by at least one of the plurality of user operations, and
a third specifying unit configured to specify an examination procedure for the current medical examination among the plurality of examination procedures in accordance with a type and ordinal position of the current user operation.

9. An image processing apparatus comprising:
a storage unit configured to store a plurality of codes and a plurality of operation image data sets in association with each other, the plurality of codes being concerned with a plurality of standard user operations in medical examination, the plurality of operation image data sets being displayed for assisting the plurality of user operations;
an operation unit configured to accept a current user operation by a user;
a first specifying unit configured to specify a specific code concerning a user operation next to the current user operation among the plurality of codes in accordance with an examination procedure for current medical examination corresponding to the current user operation;
a second specifying unit configured to specify a specific operation image data set among the plurality of operation image data sets, the specific operation image data set being concerned with the specific code; and
a display unit configured to display the specific operation image.

10. The apparatus according to claim 9, wherein the operation image data set includes at least one of a message image data set, a reduced image data set of an operation panel, an illustration image data set of the operation panel, and a component image data set, the message image data set being concerned with at least one of a problem corresponding to the current user operation and a solution for the problem, the component image data set being concerned with an electric component on the operation panel, the electric component being associated with the current user operation.

11. The apparatus according to claim 9, wherein if the specific operation image is a component image, the display unit configured to display a mark indicating a movable range of an electric component corresponding to the component image.

12. The apparatus according to claim 9, wherein the operation image data set is a flowchart image data set schematically representing an examination procedure for medical examination.

13. The apparatus according to claim 12, wherein the flowchart image data set includes a plurality of block image data sets concerning a plurality of blocks on the examination procedure.

14. The apparatus according to claim 9, further comprising an execution unit configured to execute specific processing corresponding to the specific operation image data set, when the specific operation image is selected via the operation unit.

15. The apparatus according to claim 14, which further comprises a second storage unit configured to store the plurality of operation image data sets and the plurality of processing contents in association with each other, and in which
the execution unit configured to search the second storage unit by using the specific operation image data set as a search keyword, specifies a specific processing content associated with the specific operation image data set, and executes the specific processing in accordance with the specific processing content.

16. The apparatus according to claim 9, further comprising
a third storage unit configured to store a plurality of standard examination procedures for a plurality of medical examinations, each of the plurality of examination procedures being constituted by at least one of the plurality of user operations, and
a third specifying unit configured to specify an examination procedure for the current medical examination among the plurality of examination procedures in accordance with a type and ordinal position of the current user operation.

17. A computer-readable recording medium recording a program for causing a computer to accept a current user operation,
specify a code concerning a user operation next to the current user operation among a plurality of codes concerning a plurality of user operations in accordance with an examination procedure for medical examination corresponding to the current user operation,
specify a operation image data set concerning the specified code among a plurality of operation information files representing the plurality of user operations, and
display the specified operation image.
